(19) United States
(12) Patent Application Publication
(10) Pub. No.: US 2024/0423592 A1
(43) Pub. Date: Dec. 26, 2024

(54) ULTRASOUND DIAGNOSTIC APPARATUS, CONTROL METHOD OF ULTRASOUND DIAGNOSTIC APPARATUS, AND DISTANCE MEASUREMENT DEVICE

(71) Applicant: FUJIFILM Corporation, Tokyo (JP)

(72) Inventor: Riki IGARASHI, Tokyo (JP)

(73) Assignee: FUJIFILM Corporation, Tokyo (JP)

(21) Appl. No.: 18/827,769

(22) Filed: Sep. 8, 2024

Related U.S. Application Data
(63) Continuation of application No. PCT/JP2023/005230, filed on Feb. 15, 2023.

(30) Foreign Application Priority Data
Mar. 9, 2022 (JP) 2022-036083

(51) Int. Cl. A61B 8/00; A61B 8/08

(52) U.S. Cl. CPC A61B 8/54; A61B 8/461; A61B 8/5223

(57) ABSTRACT

An ultrasound diagnostic apparatus includes: an examination position specification unit (25) that specifies an examination position of a subject by an examiner based on posture information of the examiner and the subject, which is acquired by analyzing reflection signals in a case where detection signals are transmitted from a distance measurement device (42) to the examiner and the subject; and a memory (26) that stores an ultrasound image of the subject and the examination position specified by the examination position specification unit (25) in association with each other.

### CROSS-REFERENCE TO RELATED APPLICATIONS

This application is a Continuation of PCT International Application No. PCT/JP2023/005230 filed on Feb. 15, 2023, which claims priority under 35 U.S.C. § 119(a) to Japanese Patent Application No. 2022-036083 filed on Mar. 9, 2022. The above applications are hereby expressly incorporated by reference, in their entirety, into the present application.

### BACKGROUND OF THE INVENTION

### 1. Field of the Invention

The present invention relates to an ultrasound diagnostic apparatus that specifies an examination position of a subject, a control method of the ultrasound diagnostic apparatus, and a distance measurement device.

### 2. Description of the Related Art

Conventionally, an ultrasound image representing a tomographic image in a subject has been captured by using a so-called ultrasound diagnostic apparatus. A doctor diagnoses the subject by confirming the ultrasound image. Usually, simply confirming the ultrasound image makes it difficult to determine which examination position of the subject the ultrasound image corresponds to. Therefore, work of recording the corresponding examination position is performed with respect to the ultrasound image in many cases.

In that respect, a technology for automatically determining the examination position has been developed. For example, JP2012-055774A discloses a technology for determining, in a case of examining a breast of a subject, which of left and right breasts is examined by detecting a position of an ultrasound probe using an infrared ray or a magnetic sensor.

### SUMMARY OF THE INVENTION

However, in the technology for JP2012-055774A, there is a need to register a correspondence relationship between an examination position on the subject and the position of the ultrasound probe, and there is a problem that the examination position cannot be accurately specified in a case where a posture of the subject is changed in the middle of the examination. The present invention has been made in order to solve such a conventional problem, and an object of the present invention is to provide an ultrasound diagnostic apparatus, a control method of an ultrasound diagnostic apparatus, and a distance measurement device capable of accurately specifying an examination position even in a case where a posture of a subject is changed in a middle of an examination.

The above-described object can be achieved by the following configuration.

[1] An ultrasound diagnostic apparatus comprising:
an examination position specification unit that specifies an examination position of a subject by an examiner based on posture information of the examiner and the subject, which is acquired by analyzing reflection signals in a case where detection signals are transmitted from a distance measurement device to the examiner and the subject; and
a memory that stores an ultrasound image of the subject and the examination position specified by the examination position specification unit in association with each other.

[2] The ultrasound diagnostic apparatus according to [1], further comprising:
an ultrasound probe;
an image acquisition unit that acquires the ultrasound image at the examination position of the subject by performing transmission and reception of an ultrasound beam using the ultrasound probe; and
a monitor that displays the ultrasound image.

[3] The ultrasound diagnostic apparatus according to [2], further comprising:
a control unit that displays the examination position specified by the examination position specification unit on the monitor.

[4] The ultrasound diagnostic apparatus according to [3], further comprising:
a body mark generation unit that generates a body mark indicating the examination position specified by the examination position specification unit,
in which the control unit displays the body mark on the monitor.

[5] The ultrasound diagnostic apparatus according to [4], further comprising:
a calibration unit that corrects a deviation of the examination position on the body mark caused by an individual difference in a physique of the subject.

[6] The ultrasound diagnostic apparatus according to [4] or [5], further comprising:
an input device that accepts an input operation by the examiner,
in which the body mark generation unit automatically generates the body mark indicating the examination position and displays the body mark on the monitor, in a case where a freeze operation is performed by the examiner via the input device.

[7] The ultrasound diagnostic apparatus according to any one of [3] to [6], further comprising:
a measurement unit that measures the subject at the examination position,
in which the control unit displays a measurement result by the measurement unit on the monitor.

[8] The ultrasound diagnostic apparatus according to any one of [2] to [7], further comprising:
an image acquisition condition setting unit that sets an ultrasound image acquisition condition corresponding to the examination position specified by the examination position specification unit,
in which the image acquisition unit acquires the ultrasound image in accordance with the ultrasound image acquisition condition set by the image acquisition condition setting unit.

[9] The ultrasound diagnostic apparatus according to [8],
in which the image acquisition condition setting unit selects the ultrasound image acquisition condition corresponding to the examination position specified by the examination position specification unit from among a plurality of the ultrasound image acquisition conditions preset according to a plurality of the examination positions.

[10] The ultrasound diagnostic apparatus according to [8] or [9],
in which the ultrasound image acquisition condition includes at least one of an ultrasound beam depth, a focus position, or image processing.

[11] A control method of an ultrasound diagnostic apparatus, comprising:
specifying an examination position of a subject by an examiner based on posture information of the examiner and the subject, which is acquired by analyzing reflection signals in a case where detection signals are transmitted from a distance measurement device to the examiner and the subject; and
storing an ultrasound image of the subject and the specified examination position in a memory in association with each other.

[12] A distance measurement device comprising:
a distance measurement sensing device that transmits detection signals and receives reflection signals with respect to an examiner and a subject;
a signal analysis unit that analyzes the reflection signals received by the distance measurement sensing device to acquire posture information of the examiner and the subject; and
an examination position specification unit that specifies each of the examiner and the subject and specifies an examination position of the subject by the examiner, based on the posture information acquired by the signal analysis unit.

[13] The distance measurement device according to [12],
in which the signal analysis unit acquires the posture information of the examiner and the subject by using a machine learning model that has learned a reflection signal in a case where a detection signal is transmitted to a human body by the distance measurement sensing device.

According to the present invention, there is provided an ultrasound diagnostic apparatus comprising: an examination position specification unit that specifies an examination position of a subject by an examiner based on posture information of the examiner and the subject, which is acquired by analyzing reflection signals in a case where detection signals are transmitted from a distance measurement device to the examiner and the subject; and a memory that stores an ultrasound image of the subject and the examination position specified by the examination position specification unit in association with each other. Therefore, the examination position can be accurately specified even in a case where the posture of the subject is changed in the middle of the examination.

### BRIEF DESCRIPTION OF THE DRAWINGS

FIG. 1 is a block diagram showing a configuration of an ultrasound diagnostic apparatus according to Embodiment 1 of the present invention.

FIG. 2 is a block diagram showing a configuration of a transmission and reception circuit in Embodiment 1 of the present invention.

FIG. 3 is a block diagram showing a configuration of an image generation unit in Embodiment 1 of the present invention.

FIG. 4 is a diagram schematically showing an example of a positional relationship between a distance measurement sensing device, a subject, and an examiner in Embodiment 1 of the present invention.

FIG. 5 is a flowchart showing an operation of the ultrasound diagnostic apparatus according to Embodiment 1 of the present invention.

FIG. 6 is a block diagram showing a configuration of an ultrasound diagnostic apparatus according to Embodiment 2 of the present invention.

FIG. 7 is a flowchart showing an operation of the ultrasound diagnostic apparatus according to Embodiment 2 of the present invention.

FIG. 8 is a block diagram showing a configuration of an ultrasound diagnostic apparatus according to Embodiment 3 of the present invention.

FIG. 9 is a diagram showing an example of a body mark representing a torso of the subject in Embodiment 3 of the present invention.

FIG. 10 is a diagram showing an example of a body mark representing a left breast in Embodiment 3 of the present invention.

FIG. 11 is a diagram showing an example of a body mark representing a right breast in Embodiment 3 of the present invention.

FIG. 12 is a diagram showing an example of a probe mark disposed on the body mark representing the left breast in Embodiment 3 of the present invention.

FIG. 13 is a diagram schematically showing a center line of the subject in Embodiment 3 of the present invention.

FIG. 14 is a flowchart representing an operation of the ultrasound diagnostic apparatus according to Embodiment 3 of the present invention.

FIG. 15 is a block diagram showing a configuration of an ultrasound diagnostic apparatus according to Embodiment 4 of the present invention.

FIG. 16 is a flowchart representing an operation of the ultrasound diagnostic apparatus according to Embodiment 4 of the present invention.

FIG. 17 is a flowchart representing an operation of calibration in Embodiment 4 of the present invention.

### DESCRIPTION OF THE PREFERRED EMBODIMENTS

Hereinafter, embodiments of the present invention will be described with reference to the accompanying drawings.

Although descriptions of configuration requirements to be described below are made based on a representative embodiment of the present invention, the present invention is not limited to such an embodiment.

In the present specification, a numerical range represented by “to” means a range including numerical values described before and after “to” as a lower limit value and an upper limit value.

In the present specification, “same” and “identical” include error ranges generally allowed in the technical field.

### Embodiment 1

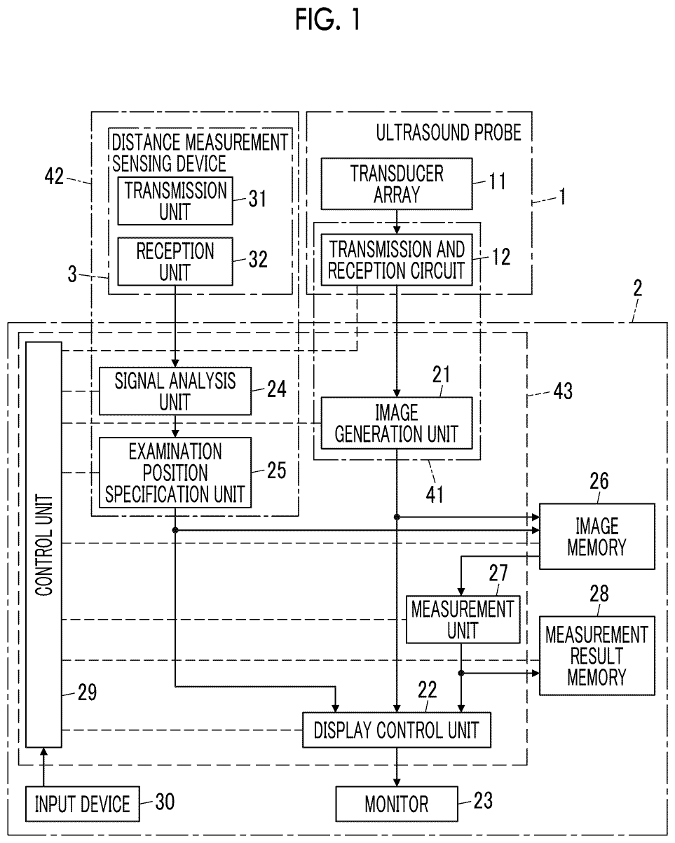

FIG. 1 shows a configuration of an ultrasound diagnostic apparatus according to Embodiment 1 of the present invention. The ultrasound diagnostic apparatus comprises an ultrasound probe 1, an apparatus body 2 connected to the ultrasound probe 1, and a distance measurement sensing device 3 connected to the apparatus body 2.

The ultrasound probe 1 includes a transducer array 11. A transmission and reception circuit 12 is connected to the transducer array 11.

The distance measurement sensing device 3 includes a transmission unit 31 and a reception unit 32.

The apparatus body 2 includes an image generation unit 21 connected to the transmission and reception circuit 12 of the ultrasound probe 1. A display control unit 22 and a monitor 23 are sequentially connected to the image generation unit 21. In addition, the apparatus body 2 includes a signal analysis unit 24 connected to the reception unit 32 of the distance measurement sensing device 3. An examination position specification unit 25 is connected to the signal analysis unit 24. In addition, an image memory 26 is connected to the image generation unit 21 and the examination position specification unit 25. Additionally, a measurement unit 27 is connected to the image memory 26. Further, a measurement result memory 28 and the display control unit 22 are connected to the measurement unit 27.

In addition, a control unit 29 is connected to the transmission and reception circuit 12, the image generation unit 21, the display control unit 22, the signal analysis unit 24, the examination position specification unit 25, the image memory 26, the measurement unit 27, and the measurement result memory 28. Further, an input device 30 is connected to the control unit 29.

In addition, the transmission and reception circuit 12 of the ultrasound probe 1 and the image generation unit 21 of the apparatus body 2 constitute an image acquisition unit 41. Further, the distance measurement sensing device 3, and the signal analysis unit 24 and the examination position specification unit 25 of the apparatus body 2 constitute a distance measurement device 42. Moreover, the image generation unit 21, the display control unit 22, the signal analysis unit 24, the examination position specification unit 25, the measurement unit 27, and the control unit 29 of the apparatus body 2 constitute a processor 43 for the apparatus body 2.

The transducer array 11 of the ultrasound probe 1 includes a plurality of ultrasound transducers arranged one-dimensionally or two-dimensionally. These ultrasound transducers each transmit an ultrasound wave in accordance with a drive signal to be supplied from the transmission and reception circuit 12, receive an ultrasound echo from a subject, and output a signal based on the ultrasound echo. For example, each ultrasound transducer is composed of a piezoelectric body and electrodes formed at both ends of the piezoelectric body. The piezoelectric body consists of a piezoelectric ceramic represented by lead zirconate titanate (PZT), a polymer piezoelectric element represented by poly vinylidene di fluoride (PVDF), a piezoelectric single crystal represented by lead magnesium niobate-lead titanate (PMN-PT), or the like.

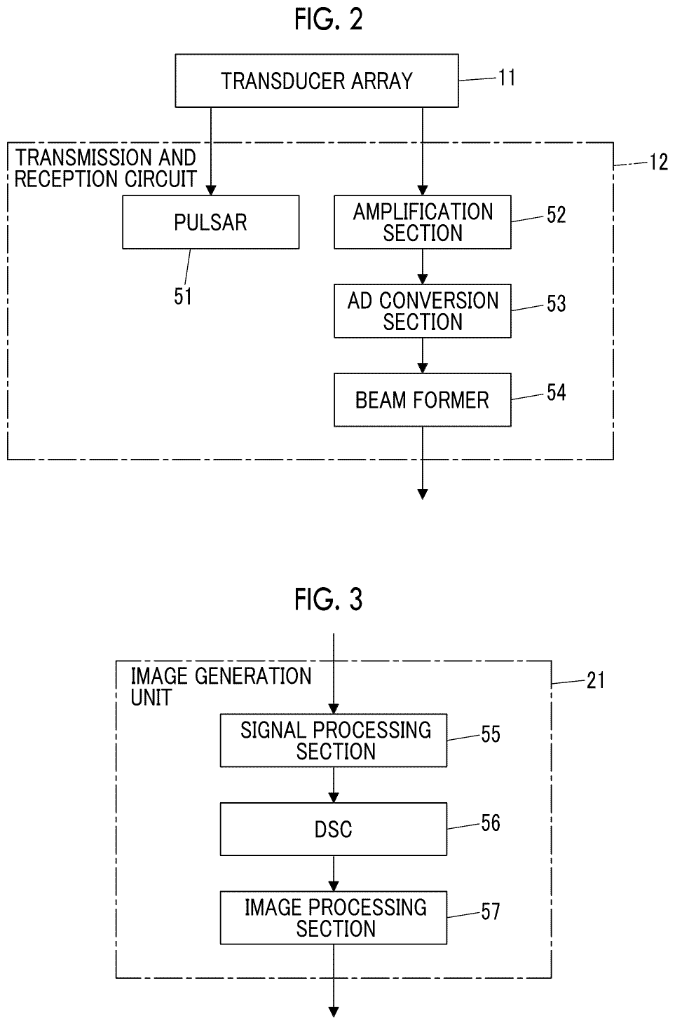

The transmission and reception circuit 12, under the control of the control unit 29, transmits the ultrasound wave from the transducer array 11 and generates a sound ray signal based on a reception signal acquired by the transducer array 11. The transmission and reception circuit 12 includes a pulsar 51 that is connected to the transducer array 11, and an amplification section 52, an analog-to-digital (AD) conversion section 53, and a beam former 54 that are sequentially connected in series from the transducer array 11, as shown in FIG. 2.

The pulsar 51 includes, for example, a plurality of pulse generators, and adjusts an amount of delay of each of drive signals and supplies the drive signals to the plurality of ultrasound transducers such that ultrasound waves transmitted from the plurality of ultrasound transducers of the transducer array 11 form an ultrasound beam, based on a transmission delay pattern selected according to a control signal from the control unit 29. In this manner, in a case where a pulsed or continuous wave-like voltage is applied to the electrodes of the ultrasound transducer of the transducer array 11, the piezoelectric body expands and contracts to generate a pulsed or continuous wave-like ultrasound wave from each of the ultrasound transducers, thereby forming an ultrasound beam from the combined wave of these ultrasound waves.

The transmitted ultrasound beam is reflected in, for example, a target such as a site of the subject and propagates toward the transducer array 11 of the ultrasound probe 1. The ultrasound echo that propagates toward the transducer array 11 in this manner is received by each of the ultrasound transducers that constitute the transducer array 11. In this case, each of the ultrasound transducers that constitute the transducer array 11 receives the propagating ultrasound echo to expand and contract to generate a reception signal which is an electrical signal, thereby outputting these reception signals to the amplification section 52.

The amplification section 52 amplifies the signal input from each of the ultrasound transducers that constitute the transducer array 11 and transmits the amplified signal to the AD conversion section 53. The AD conversion section 53 converts the signal transmitted from the amplification section 52 into digital reception data. The beam former 54 performs so-called reception focus processing by applying and adding a delay to each reception data received from the AD conversion section 53. Through the reception focus processing, the sound ray signal in which each reception data converted by the AD conversion section 53 is phase-added and a focus of the ultrasound echo is narrowed down is acquired.

As shown in FIG. 3, the image generation unit 21 has a configuration in which a signal processing section 55, a digital scan converter (DSC) 56, and an image processing section 57 are sequentially connected in series.

The signal processing section 55 generates a B-mode image signal, which is tomographic image information regarding tissues inside the subject, by performing, on the sound ray signal received from the transmission and reception circuit 12, envelope detection processing after performing correction of attenuation due to a distance according to a depth of a reflection position of the ultrasound wave using a sound velocity value set by the control unit 29.

The DSC 56 converts (raster-converts) the B-mode image signal generated by the signal processing section 55 into an image signal following a normal television signal scanning method.

The image processing section 57 performs various types of necessary image processing such as gradation processing on the B-mode image signal to be input from the DSC 56, and then sends the B-mode image signal to the display control unit 22 and the image memory 26. Hereinafter, the B-mode image signal that has been subjected to the image processing by the image processing section 57 will be referred to as an ultrasound image.

The display control unit 22, under the control of the control unit 29, performs predetermined processing on the ultrasound image or the like generated by the image generation unit 21 and displays the ultrasound image or the like on the monitor 23.

The monitor 23 performs various types of display under the control of the display control unit 22. Examples of the monitor 23 include a display device such as a liquid crystal display (LCD) or an organic electroluminescence (EL) display.

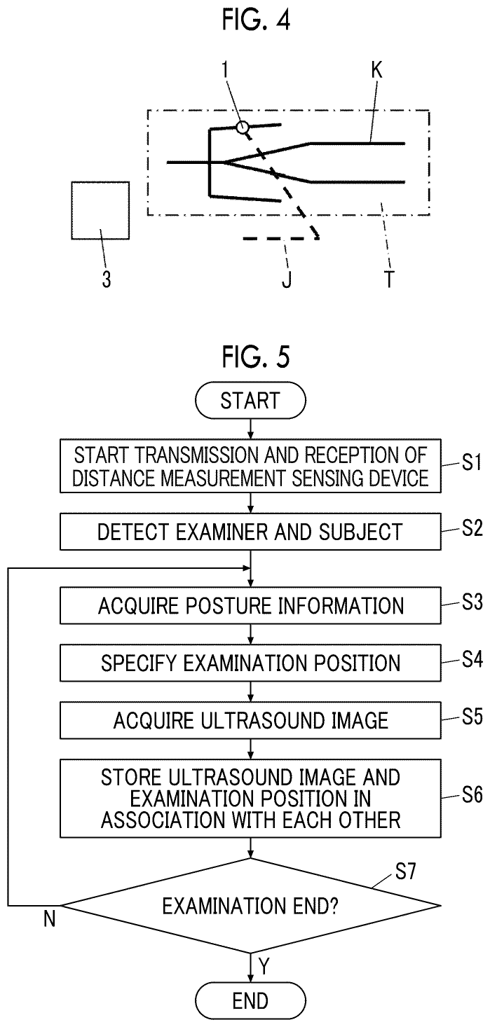

For example, as shown in FIG. 4, the distance measurement sensing device 3 is disposed near a subject K and an examiner J who performs an examination for the subject K by using the ultrasound diagnostic apparatus, and transmits detection signals to the examiner J and the subject K and receives reflection signals from the examiner J and the subject K. In the example of FIG. 4, a situation is depicted in which the subject K is lying on an examination table T, and the examiner J examines an arm part of the subject K with the ultrasound probe 1.

The transmission unit 31 of the distance measurement sensing device 3 transmits the detection signals to the examiner J and the subject K. The transmission unit 31 is a so-called radio transmitter for electromagnetic waves and includes, for example, an antenna for transmitting electromagnetic waves, a signal source such as an oscillation circuit, a modulation circuit for modulating signals, an amplifier for amplifying signals, and the like.

The reception unit 32 includes an antenna for receiving electromagnetic waves and the like and receives the reflection signals from the examiner J and the subject K.

The distance measurement sensing device 3 can be configured with, for example, a radar that transmits and receives so-called Wi-Fi (registered trademark) standard detection signals consisting of electromagnetic waves having a center frequency of 2.4 GHz or 5 GHz and can also be configured with a radar that transmits and receives wideband detection signals having a center frequency of 1.78 GHZ. In addition, the distance measurement sensing device 3 can also be configured with a so-called light detection and ranging or laser imaging detection and ranging (LIDAR) sensor that transmits short-wavelength electromagnetic waves such as ultraviolet rays, visible rays, or infrared rays as detection signals.

The signal analysis unit 24 of the apparatus body 2 acquires posture information of the examiner J and the subject K by analyzing the reflection signals received by the distance measurement sensing device 3. The posture information of the examiner J and the subject K includes, for example, information regarding a position of each site of the examiner J and the subject K such as head parts, shoulder parts, arm parts, waist parts, and leg parts of the examiner J and the subject K.

The signal analysis unit 24 can acquire the posture information of the examiner J and the subject K by using a machine learning model that has learned a reflection signal in a case where a detection signal is transmitted to a human body by the distance measurement sensing device 3. Specifically, the signal analysis unit 24 can acquire the posture information by using, for example, a method described in “ZHAO, Mingmin, et al., Through-wall human pose estimation using radio signals, In: Proceedings of the IEEE Conference on Computer Vision and Pattern Recognition, 2018, pp. 7356 to 7365”, “VASILEIADIS, Manolis; BOUGANIS, Christos-Savvas; TZOVARAS, Dimitrios, Multi-person 3D pose estimation from 3D cloud data using 3D convolutional neural networks, Computer Vision and Image Understanding, 2019, 185:12 to 23”, “JIANG, Wenjun, et al., Towards 3D human pose construction using WiFi, In: Proceedings of the 26th Annual International Conference on Mobile Computing and Networking, 2020, pp. 1 to 14”, or “WANG, Fei, et al., Person-in-WiFi: Fine-grained person perception using WiFi, In: Proceedings of the IEEE/CVF International Conference on Computer Vision, 2019, pp. 5452 to 5461”.

The examination position specification unit 25 specifies each of the examiner J and the subject K and specifies the examination position of the subject K by the examiner J, based on the posture information acquired by the signal analysis unit 24. The examination position specification unit 25 can specify, for example, the position of the examiner J's fingertip based on the posture information and can specify the specified position of the fingertip as the examination position by the ultrasound probe 1. The examination position specification unit 25 can refer to, for example, the posture information to specify a person in a posture of lying down as the subject K and specify a person in a posture of touching the specified subject K as the examiner J.

In a case where the examination position specification unit 25 has failed to specify the examiner J or the subject K for some reason, the examination position specification unit 25 can perform the processing of specifying the examiner J and the subject K again in response to an instruction by the examiner via the input device 30.

Here, the examination position specification unit 25 can specify, for example, a relative position between the subject K and the examiner J, which is represented by using coordinates, as the examination position. In addition, the examination position specification unit 25 can also specify, for example, organs such as the left breast, the right breast, the left lung, the right lung, or the heart as the examination position. Further, the examination position specification unit 25 can also specify, for example, sites larger than the organs, such as an abdomen or an upper limb, as the examination position. Moreover, the examination position specification unit 25 can also convert and output the specified examination position into information such as a numerical value or a code name corresponding to the examination position, in addition to the coordinates or the name of the examination position.

In addition, the examination position specification unit 25 can also send the specified examination position to the display control unit 22 and display the examination position on the monitor 23 together with the ultrasound image generated by the image generation unit 21.

The image memory 26 stores the ultrasound image generated by the image generation unit 21 and the examination position of the subject K specified by the examination position specification unit 25 in association with each other under the control of the control unit 29. The image memory 26 can associate the ultrasound image and the examination position with each other, for example, by describing the examination position in so-called header information of the ultrasound image, under the control of the control unit 29. Further, the image memory 26 can also associate the ultrasound image and the examination position with each other by using, for example, a so-called time stamp or so-called Digital Imaging and Communications in Medicine (DICOM), under the control of the control unit 29.

As the image memory 26, for example, recording media such as a flash memory, a hard disk drive (HDD), a solid state drive (SSD), a flexible disk (FD), a magneto-optical disk (MO disk), a magnetic tape (MT), a random access memory (RAM), a compact disc (CD), a digital versatile disc (DVD), a secure digital card (SD card), or a universal serial bus memory (USB memory), and the like can be used.

The measurement unit 27, under the control of the control unit 29, reads out the ultrasound image stored in the image memory 26 and performs the measurement of the subject K at the examination position corresponding to the ultrasound image based on the read-out ultrasound image. The measurement unit 27 can measure, for example, dimensions or the like of anatomical structures in blood vessels appearing in the ultrasound image based on an input operation by the examiner J via the input device 30.

The measurement result memory 28, under the control of the control unit 29, stores a result measured by the measurement unit 27 in association with the ultrasound image used for the measurement. As the measurement result memory 28, for example, recording media such as a flash memory, an HDD, an SSD, an FD, an MO disk, an MT, a RAM, a CD, a DVD, an SD card, or a USB memory, and the like can be used.

The input device 30 accepts the input operation by the examiner J and sends input information to the control unit 29. The input device 30 is composed of, for example, a device for the examiner J to perform an input operation such as a keyboard, a mouse, a trackball, a touchpad, or a touch panel.

Although the processor 43 including the image generation unit 21, the display control unit 22, the signal analysis unit 24, the examination position specification unit 25, the measurement unit 27, and the control unit 29 of the apparatus body 2 is configured with a central processing unit (CPU) and a control program for causing the CPU to perform various types of processing, the processor 43 may be configured by using a field programmable gate array (FPGA), a digital signal processor (DSP), an application specific integrated circuit (ASIC), a graphics processing unit (GPU), or other integrated circuits (ICs), or may be configured with a combination thereof.

In addition, the image generation unit 21, the display control unit 22, the signal analysis unit 24, the examination position specification unit 25, the measurement unit 27, and the control unit 29 of the processor 43 can also be configured by being integrated partially or entirely into one CPU or the like.

Next, an example of the operation of the ultrasound diagnostic apparatus according to Embodiment 1 will be described using the flowchart of FIG. 5.

First, in step S1, the distance measurement sensing device 3 starts the continuous transmission of the detection signals to the examiner J and the subject K and the continuous reception of the reflection signals from the examiner J and the subject K. In addition, in this case, the examiner J brings the ultrasound probe 1 into contact with the examination position of the subject K.

Next, in step S2, the signal analysis unit 24 detects the subject K and the examiner J by analyzing the reflection signals received by the distance measurement sensing device 3 in step S1.

In subsequent step S3, the signal analysis unit 24 acquires the posture information of the subject K and the examiner J detected in step S2 by analyzing the reflection signals received by the distance measurement sensing device 3 in step S1. The signal analysis unit 24 sends the acquired posture information to the examination position specification unit 25.

In step S4, the examination position specification unit 25 specifies the examination position of the subject K by the examiner J based on the posture information acquired in step S3. In this case, the examination position specification unit 25 can specify, for example, the position of the examiner J's fingertip based on the posture information and can specify the specified position of the fingertip as the examination position by the ultrasound probe 1.

As described above, in steps S1 to S4, the posture information of the examiner J and the subject K is acquired by analyzing the reflection signals received by the distance measurement sensing device 3, and the examination position of the subject K is specified based on the acquired posture information. Therefore, the examination position of the subject K can be accurately specified even in a case where the posture of the subject K is changed during the examination.

In step S5 following step S4, the inside of the subject K is scanned by the ultrasound probe 1, and the ultrasound image representing the tomographic image in the subject K is acquired. In this case, the transmission and reception circuit 12 performs so-called reception focus processing to generate the sound ray signal, under the control of the control unit 29. The sound ray signal generated by the transmission and reception circuit 12 is sent to the image generation unit 21. The image generation unit 21 generates the ultrasound image by using the sound ray signal sent from the transmission and reception circuit 12.

The ultrasound image acquired in such a manner is sent to the display control unit 22 and the image memory 26. The ultrasound image sent to the display control unit 22 is displayed on the monitor 23 after being subjected to predetermined processing.

In step S6, the image memory 26, under the control of the control unit 29, stores the ultrasound image acquired in step S5 and the examination position of the subject K specified in step S4 in association with each other.

As described above, the ultrasound image and the corresponding examination position are automatically associated with each other and stored in the image memory 26, so that, for example, there is no need for the examiner J to manually associate the ultrasound image with the examination position, and the ultrasound image and the examination position can be easily and accurately associated with each other.

In addition, in such a manner, by storing the ultrasound image and the corresponding examination position in the image memory 26 in association with each other, for example, in a case where the doctor confirms the ultrasound image after the examination and performs the diagnosis on the subject K, the doctor can easily understand the examination position corresponding to the ultrasound image, and the diagnosis can be smoothly performed.

In subsequent step S7, the control unit 29 determines whether or not to end the examination. For example, in a case where instruction information to end the examination is input by the examiner J via the input device 30, the control unit 29 determines to end the current examination. Alternatively, for example, in a case where no instruction information to end the ultrasound examination is input by the examiner J via the input device 30, it is determined to continue the current examination.

In a case where it is determined in step S7 to continue the examination, the processing returns to step S3. As described above, the processing of steps S3 to S7 is repeated as long as it is determined in step S7 to continue the examination.

In addition, in a case where it is determined to end the examination in step S7, each unit of the ultrasound diagnostic apparatus is controlled by the control unit 29 so as to end the examination, and the operation of the ultrasound diagnostic apparatus following the flowchart of FIG. 5 ends.

As described above, with the ultrasound diagnostic apparatus according to Embodiment 1 of the present invention, the examination position specification unit 25 specifies the examination position of the subject K by the examiner J by analyzing the posture information acquired by the signal analysis unit 24 based on the reflection signals received by the distance measurement sensing device 3, so that the examination position of the subject K can be accurately specified even in a case where the posture of the subject K is changed during the examination. In addition, since the image memory 26 stores the ultrasound image of the subject K and the examination position specified by the examination position specification unit 25 in association with each other, for example, there is no need for the examiner J to manually associate the ultrasound image with the examination position, and the ultrasound image and the examination position can be easily and accurately associated with each other.

Further, with the ultrasound diagnostic apparatus according to Embodiment 1 of the present invention, for example, there is no need to capture the optical image of the subject K in order to specify the examination position of the subject K, so that the examination position can be specified while ensuring the privacy of the subject K.

The image generation unit 21 has been described as being provided in the apparatus body 2, but the image generation unit 21 can also be provided in the ultrasound probe 1 instead of being provided in the apparatus body 2.

In addition, the signal analysis unit 24 has been described as being provided in the apparatus body 2, but for example, the distance measurement sensing device 3 and the signal analysis unit 24 can also constitute the distance measurement device 42 independent of the apparatus body 2. In this case, the posture information of the examiner J and the subject K are acquired by the signal analysis unit 24 of the distance measurement device 42, and the acquired posture information is sent to the examination position specification unit 25 of the apparatus body 2. Therefore, in this case as well, the examination position of the subject K is specified by the examination position specification unit 25, and the specified examination position is stored in the image memory 26 in association with the ultrasound image, similar to a case where the apparatus body 2 comprises the signal analysis unit 24.

In addition, the distance measurement sensing device 3, the signal analysis unit 24, and the examination position specification unit 25 can also constitute the distance measurement device 42 independent of the apparatus body 2. In this case, the posture information is acquired in the distance measurement device 42, the examination position of the subject K is specified based on the posture information, and the specified examination position is sent to the image memory 26 of the apparatus body 2. Therefore, in this case as well, the specified examination position is stored in the image memory 26 in association with the ultrasound image, similar to a case where the apparatus body 2 comprises the signal analysis unit 24 and the examination position specification unit 25.

Further, for example, FIG. 4 shows that the distance measurement sensing device 3 is installed near the examiner J and the subject K, but the installation position of the distance measurement sensing device 3 is not particularly limited as long as the detection signals to be transmitted from the distance measurement sensing device 3 reach the examiner J and the subject K. For example, the distance measurement sensing device 3 can also be installed on the ceiling of the room where the examiner J performs the examination for the subject K.

In addition, the examination position specification unit 25 stores, for example, the initial position of the subject K even in a case where the detection signal is obstructed by the examiner J during the examination and does not reach the subject K, whereby the examination position of the subject K can also be estimated based on the posture information of the examiner J and the subject K.

Further, in the flowchart of FIG. 5, the processing proceeds in the order of steps S3, S4, and S5, but steps S3, S4, and S5 can also be processed in parallel.

In addition, in a case where the processing of steps S3 to S7 is repeatedly performed, the control unit 29 can skip step S4 in a case where the posture information acquired in step S3 is substantially the same as the posture information acquired in previously performed step S3, through the comparison of the posture information. In this case, the control unit 29 can perform, for example, processing such as matching between the currently acquired postures of the subject K and the examiner J and the previously acquired postures of the subject K and the examiner J and can calculate the degree of similarity between the postures. The control unit 29 can determine that the currently acquired posture information and the previously acquired posture information are substantially the same, for example, in a case where the calculated degree of similarity is equal to or greater than a certain threshold value. In addition, in a case where the processing of step S4 is skipped, in step S6, the ultrasound image acquired in the current step S5 and the examination position specified in the previous step S4 are stored in the image memory 26 in association with each other.

Further, in the flowchart of FIG. 5, the ultrasound image has been described as being acquired in step S5 each time the posture information is acquired in step S3, but for example, the posture information can also be acquired once in step S3 each time ultrasound images of a plurality of constant frames are acquired in step S5. Additionally, the ultrasound image of a single frame can also be acquired in step S5 each time the posture information is acquired a plurality of times in step S3.

In addition, in the flowchart of FIG. 5, measurement processing by the measurement unit 27 can also be added. For example, after the ultrasound image and the examination position are stored in the image memory 26 in association with each other in step S6, the measurement by the measurement unit 27 can be performed. In this case, the measurement unit 27 can read out the ultrasound image stored in step S6 from the image memory 26 and measure the dimensions or the like of the anatomical structures in the ultrasound image based on an input operation by the examiner J via the input device 30. A measurement result obtained by the measurement unit 27 in such a manner is stored in the measurement result memory 28.

In addition, examination protocols including a plurality of predetermined examination positions are generally known, such as so-called extended focused assessment with sonography for trauma (eFAST). In a case where the examination is performed in accordance with such examination protocols, the control unit 29 determines, for example, whether or not all the examinations of all the examination positions included in the examination protocols have ended, and in a case where all the examinations of all the examination positions have not ended, the unexamined examination site can be displayed on the monitor 23. In this case, the control unit 29 can determine that the examination at the examination position has been completed, for example, in a case where the ultrasound image and the examination position are stored in the image memory 26 in association with each other in step S6. In such a manner, by displaying the unexamined examination site on the monitor 23, the examiner J can easily understand whether or not all the examination positions have already been examined, and can perform the examination without omission.

### Embodiment 2

The ultrasound diagnostic apparatus can also acquire the ultrasound image by using an appropriate condition for the examination position of the subject K specified by the examination position specification unit 25.

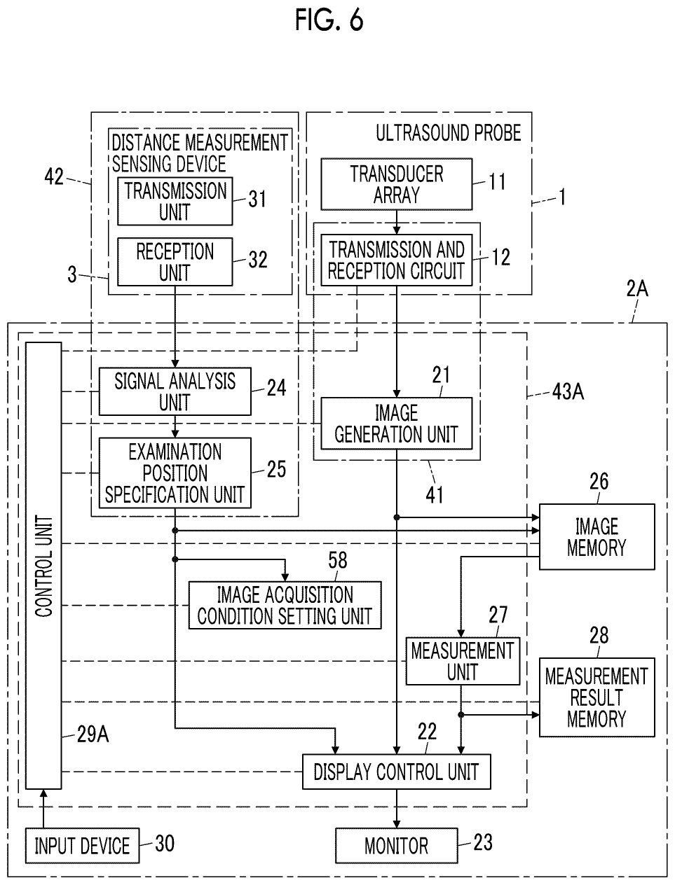

FIG. 6 shows a configuration of an ultrasound diagnostic apparatus according to Embodiment 2 of the present invention. The ultrasound diagnostic apparatus of Embodiment 2 comprises an apparatus body 2A instead of the apparatus body 2 with respect to the ultrasound diagnostic apparatus of Embodiment 1. In the apparatus body 2A in Embodiment 2, an image acquisition condition setting unit 58 is added, and a control unit 29A is provided instead of the control unit 29 with respect to the apparatus body 2 in Embodiment 1.

In the apparatus body 2A, the image acquisition condition setting unit 58 is connected to the examination position specification unit 25 and the control unit 29A. In addition, the image generation unit 21, the display control unit 22, the signal analysis unit 24, the examination position specification unit 25, the measurement unit 27, the control unit 29A, and the image acquisition condition setting unit 58 constitute a processor 43A for the apparatus body 2A.

The image acquisition condition setting unit 58 sets an ultrasound image acquisition condition corresponding to the examination position specified by the examination position specification unit 25. The ultrasound image acquisition condition is various conditions set in a case of acquiring the ultrasound image and includes, for example, a so-called ultrasound beam depth, a so-called focus position, and a parameter of image processing, such as a brightness and a gain. For example, in a case where the examination position specified by the examination position specification unit 25 corresponds to the lung of the subject K, the image acquisition condition setting unit 58 can set the ultrasound image acquisition condition corresponding to the lung such that the lung of the subject K can be clearly imaged.

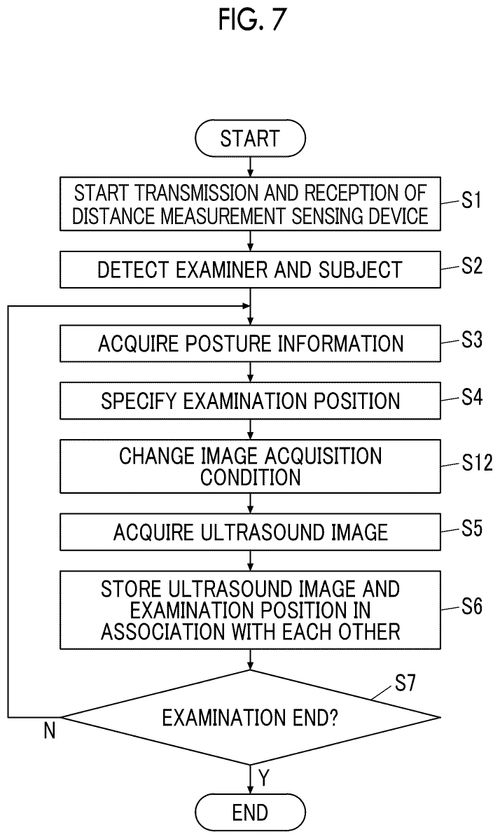

Here, the operation of the ultrasound diagnostic apparatus of Embodiment 2 will be described with reference to the flowchart of FIG. 7. The flowchart of FIG. 7 is a flowchart in which step S12 is added between steps S4 and S5 with respect to the flowchart of FIG. 5 in Embodiment 1. Therefore, detailed descriptions of steps S1 to S7 will not be repeated.

In a case where the examination position of the subject K is specified by the examination position specification unit 25 in step S4, the process proceeds to step S12.

In step S12, the image acquisition condition setting unit 58 sets the ultrasound image acquisition condition corresponding to the examination position of the subject K specified in step S4. For example, in a case where the examination position specified in step S4 corresponds to the lung of the subject K, the image acquisition condition setting unit 58 can set the ultrasound image acquisition condition corresponding to the lung such that the lung of the subject K can be clearly imaged.

In step S5 following step S12, the ultrasound image is acquired in accordance with the ultrasound image acquisition condition set in step S12. As a result, it is possible to acquire an ultrasound image in which a site of the subject K corresponding to the examination position specified in step S4 is clearly depicted.

As described above, with the ultrasound diagnostic apparatus of Embodiment 2, the image acquisition condition setting unit 58 automatically sets the ultrasound image acquisition condition according to the examination position specified by the examination position specification unit 25, so that an appropriate ultrasound image acquisition condition corresponding to the examination position can be easily set, and an ultrasound image in which the site of the subject K to be targeted for the examination is clearly depicted can be easily acquired.

By storing in advance a plurality of ultrasound image acquisition conditions corresponding to a plurality of examination positions as so-called presets, the image acquisition condition setting unit 58 can also select the ultrasound image acquisition condition corresponding to the examination position specified by the examination position specification unit 25, from among the plurality of ultrasound image acquisition conditions preset according to the plurality of examination positions. The image acquisition condition setting unit 58 can store in advance, for example, three ultrasound image acquisition conditions corresponding to the lung, the heart, and the abdomen of the subject K, as presets. As a result, the image acquisition condition setting unit 58 can easily set the ultrasound image acquisition condition corresponding to the examination position specified by the examination position specification unit 25, and an ultrasound image in which the site of the subject K to be targeted for the examination is clearly depicted can be easily acquired.

### Embodiment 3

In general, a so-called body mark imitating a part of the body of the subject is often used in order to indicate the examination position. Usually, the examiner often manually sets an appropriate body mark corresponding to the examination position, but the body mark corresponding to the examination position specified by the examination position specification unit 25 can be automatically set.

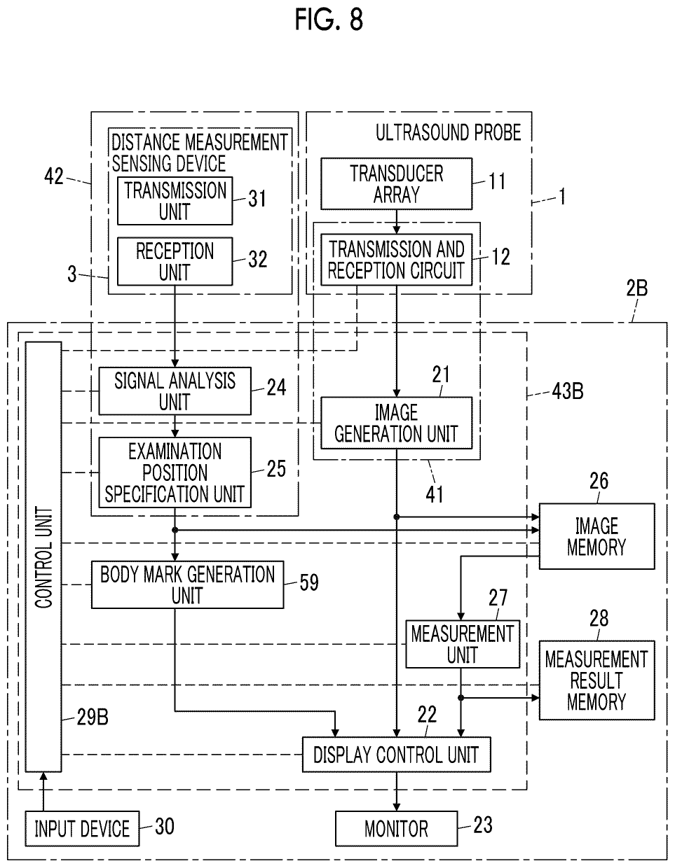

FIG. 8 shows a configuration of an ultrasound diagnostic apparatus of Embodiment 3. The ultrasound diagnostic apparatus of Embodiment 3 comprises an apparatus body 2B instead of the apparatus body 2 with respect to the ultrasound diagnostic apparatus of Embodiment 1 shown in FIG. 1. In the apparatus body 2B, a body mark generation unit 59 is added, and a control unit 29B is provided instead of the control unit 29, with respect to the apparatus body 2 in Embodiment 1.

In the apparatus body 2B, the body mark generation unit 59 is connected to the examination position specification unit 25 and the control unit 29B. In addition, the display control unit 22 is connected to the body mark generation unit 59. Further, the image generation unit 21, the display control unit 22, the signal analysis unit 24, the examination position specification unit 25, the measurement unit 27, the control unit 29B, and the body mark generation unit 59 constitute a processor 43B for the apparatus body 2B.

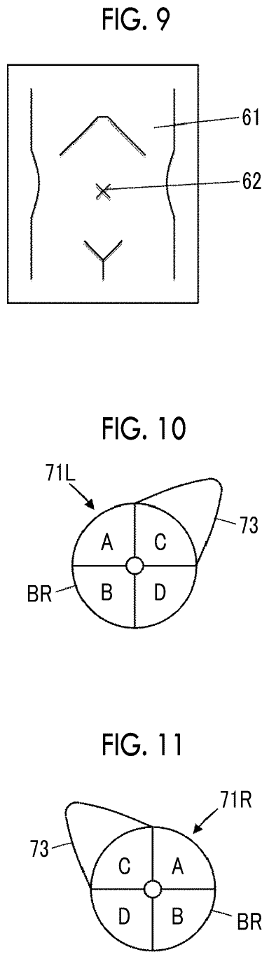

The body mark generation unit 59 generates a body mark indicating the examination position specified by the examination position specification unit 25. For example, as shown in FIG. 9, the body mark generation unit 59 can generate a body mark 61 imitating the torso of the subject K and can indicate an examination position 62 specified by the examination position specification unit 25 on the body mark 61.

In addition, in general, as shown in FIGS. 10 and 11, a body mark 71L indicating the left breast of the subject K and a body mark 71R indicating the right breast of the subject K are known.

The body mark 71L schematically indicates the left breast as viewed from the front and has a circular breast region BR and a substantially triangular axillary region 73 representing the axilla and extending diagonally upward from the breast region BR. The breast region BR is divided into four regions, that is, an inner upper region A, an inner lower region B, an outer upper region C, and an outer lower region D of the breast, and the axillary region 73 is connected to a left diagonal upper part of the outer upper region C.

The body mark 71R schematically indicates the right breast as viewed from the front and is obtained by horizontally reversing the body mark 71L indicating the left breast.

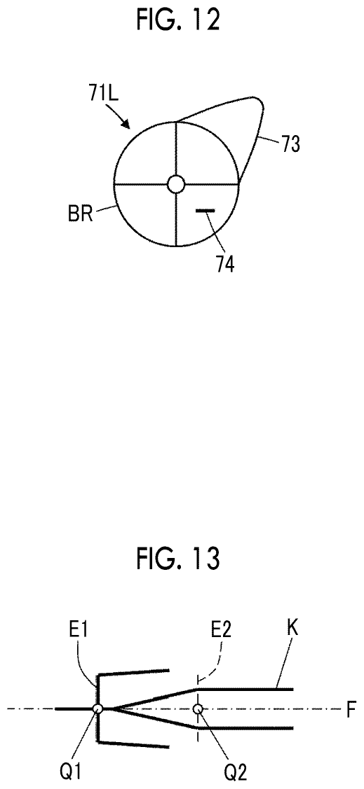

The body mark generation unit 59 can also generate, for example, the body marks 71L and 71R indicating the breasts of the subject K as shown in FIGS. 10 and 11. In this case, the body mark generation unit 59 can indicate an examination position 74 specified by the examination position specification unit 25 on the body mark 71L, for example, as shown in FIG. 12, based on an input operation by the examiner J via the input device 30. In the example of FIG. 12, the examination position 74 is shown on the outer lower region D of the body mark 71L.

In addition, in a case where the examination of the breast of the subject K is performed, the body mark generation unit 59 determines which of the left and right breasts of the subject K is examined, based on the posture information of the examiner J and the subject K acquired by the signal analysis unit 24 and stored in the image memory 26.

In this case, for example, as shown in FIG. 13, the body mark generation unit 59 calculates a center line F of the body of the subject K passing through a midpoint Q1 of the width of a shoulder part E1 and a midpoint Q2 of the width of a waist part E2 of the subject K based on the posture information of the subject K and determines whether the examiner J's fingertip is located on the right side or on the left side with respect to the center line F calculated in a case where the subject K is viewed from the front. As a result, the body mark generation unit 59 can determine whether the left breast of the subject K is examined or the right breast is examined.

The body mark generation unit 59 can generate any of the body mark 71L indicating the left breast or the body mark 71R indicating the right breast based on the information indicating which of the left and right breasts is examined, which is specified in such a manner.

The control unit 29B displays the body mark 61, 71L, or 71R generated by the body mark generation unit 59 on the monitor 23.

The measurement unit 27 measures dimensions or the like of the lesion depicted in the ultrasound image based on an input operation or the like by the examiner J via the input device 30.

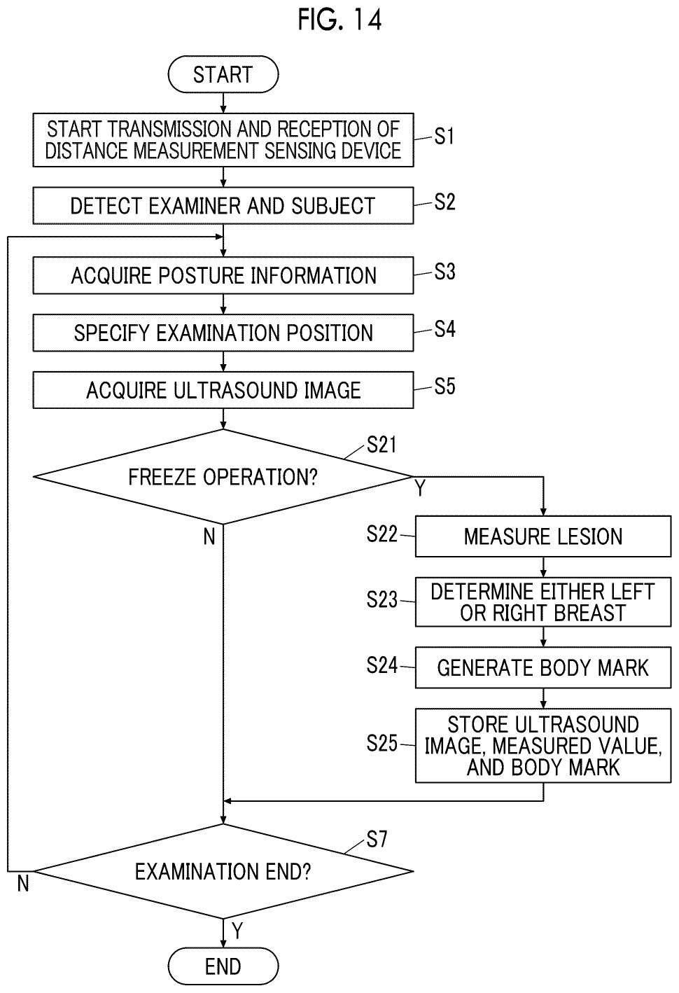

FIG. 14 shows an example of the operation of the ultrasound diagnostic apparatus of Embodiment 3 in a case of examining the breast of the subject K. The flowchart of FIG. 14 is a flowchart in which steps S21 to S25 are added instead of step S6 with respect to the flowchart of Embodiment 1 shown in FIG. 5. Since steps S1 to S7 are the same as steps S1 to S7 in Embodiment 1, detailed descriptions thereof will not be repeated.

In step S4, the examination position specification unit 25 specifies the breast of the subject K as the examination position without distinguishing between the left and right based on the posture information acquired in step S3.

In step S5, the ultrasound image is acquired.

In a case where the ultrasound image is acquired in step S5, the process proceeds to step S21. In step S21, the control unit 29B determines whether or not a freeze operation is performed by the examiner J via the input device 30. The freeze operation is an operation of freezing the ultrasound image. Freezing the ultrasound image means that an ultrasound image of a latest single frame is displayed on the monitor 23 as a still image from a state in which the ultrasound images are continuously acquired and sequentially displayed on the monitor 23. The freeze operation is performed by the examiner J via the input device 30, and the control unit 29B proceeds to step S22 in a case where it is determined that the freeze operation is performed.

In step S22, the measurement unit 27 measures the dimension or the like of the lesion depicted in the ultrasound image of the single frame frozen in step S21 based on an input operation or the like by the examiner J via the input device 30.

In subsequent step S23, the body mark generation unit 59 determines whether the breast of the subject K currently being examined, that is, the breast of the subject K corresponding to the ultrasound image frozen on the monitor 23, is either the left or right breast, based on the position information of the examiner J and the subject K stored in the image memory 26. For example, as shown in FIG. 14, the body mark generation unit 59 calculates the center line F of the body of the subject K and determines whether the Examiner J's fingertip is located on the right side or on the left side with respect to the center line F in a case where the subject K is viewed from the front, whereby it can be determined whether the breast of the subject K currently being examined is either the left breast or the right breast.

In step S24, the body mark generation unit 59 generates the body mark 71L indicating the left breast of the subject K or the body mark 71R indicating the right breast based on the determination result in step S23.

As described above, the body mark generation unit 59 automatically generates the body mark 71L or 71R corresponding to the examination position of the subject K, so that the examiner J can save the effort of manually setting the body mark 71L or 71R.

In addition, assuming that the left and right breast determinations are made for each of the ultrasound images that are continuously generated and displayed on the monitor 23, and the body mark 71L or 71R is generated, the body mark 71L imitating the left breast and the body mark 71R imitating the right breast are frequently switched, making it difficult for the examiner to easily understand the examination site. In the flowchart of FIG. 14, since the body mark 71L or 71R is generated in step S25 for the ultrasound image in a state in which the freeze operation is performed in step S21, that is, for the ultrasound image displayed in a frozen manner, the body mark 71L or 71R is stably displayed on the monitor 23. Therefore, the examiner can easily understand the current examination site.

Here, in general, since the body mark 71L indicating the left breast of the subject K and the body mark 71R indicating the right breast of the subject K have similar shapes to each other, in a case where the examiner J manually selects any of the body marks 71L and 71R to be manually indicated via the input device of the ultrasound diagnostic apparatus, the body mark 71L or 71R may be incorrectly selected.

Since the body mark generation unit 59 automatically determines which of the left and right breasts of the subject K is examined, the body mark 71L or 71R is prevented from being incorrectly selected.

In step S25, the ultrasound image frozen in step S21, the measured value of the lesion obtained in step S22, and the body mark 71L or 71R generated in step S24 are stored in the measurement result memory 28. In this case, for example, as shown in FIG. 12, the detailed examination position 74 on the body mark 71L can be recorded by the examiner J via the input device 30.

In a case where the processing of step S25 is completed in such a manner, the process proceeds to step S7.

In addition, in a case where it is determined in step S21 that the freeze operation is not performed, the process proceeds to step S7.

As described above, with the ultrasound diagnostic apparatus of Embodiment 3, the body mark generation unit 59 automatically generates the body mark 61, 71L, or 71R corresponding to the examination position of the subject K specified by the examination position specification unit 25, so that it is possible for the examiner J to save the effort of manually setting the body mark 61, 71L, or 71R, and it is possible to easily associate the body mark 61, 71L, or 71R with the ultrasound image.

Further, particularly, in a case of examining the breast of the subject K, the body mark generation unit 59 automatically determines which of the left and right breasts of the subject K is examined, so that the body mark 71L indicating the left breast of the subject K and the body mark 71R indicating the right breast can be accurately selected, and the doctor can perform a more accurate diagnosis in a case of diagnosing the subject K after the examination.

Although the aspect of Embodiment 3 has been described as being applied to the aspect of Embodiment 1, the aspect of Embodiment 3 can also be applied to the aspect of Embodiment 2 in the same manner. In this case, an appropriate ultrasound image acquisition condition corresponding to the examination position of the subject K is automatically set by the image acquisition condition setting unit 58, and the body mark 61, 71L, or 71R corresponding to the examination position of the subject K is automatically set by the body mark generation unit 59.

In addition, the breast has been exemplified as the examination position in a case where the body mark generation unit 59 determines the left and right sides of the subject K, but the examination position is not particularly limited as long as it is a site present at the left-right symmetrical position. For example, even in a case where the lung or the like of the subject K is examined, the body mark generation unit 59 can determine whether the examination position is on the left side or on the right side of the subject K.

In addition, in the flowchart of FIG. 14, instead of performing the processing of step S23 between steps S22 and S24, for example, the processing of step S23 can also be performed between steps S4 and S5.

Further, in the flowchart of FIG. 14, the processing of steps S22 to S25 is performed in a case where the freeze operation is performed in step S21, but for example, step S21 can be skipped, and the process can also proceed to step S22 after the ultrasound image is acquired in step S5. In this case, each time the ultrasound image is acquired in step S5, the processing of measuring the lesion in step S22 in real time, the processing of determining the left and right breasts in step S23, the processing of generating the body mark 71L or 71R in step S24, and the processing of storing the ultrasound image, the measured value, and the body mark 71L or 71R in step S25 are performed.

### Embodiment 4

In a case of examining the breast of the subject K, in Embodiment 3, it has been described that the examination position is manually input by the examiner J via the input device 30 on the body mark 71L or 71R indicating the breast of the subject K, but the examination position can also be automatically and accurately input on the body mark imitating the specific site of the subject K, such as the body mark 71L or 71R indicating the breast.

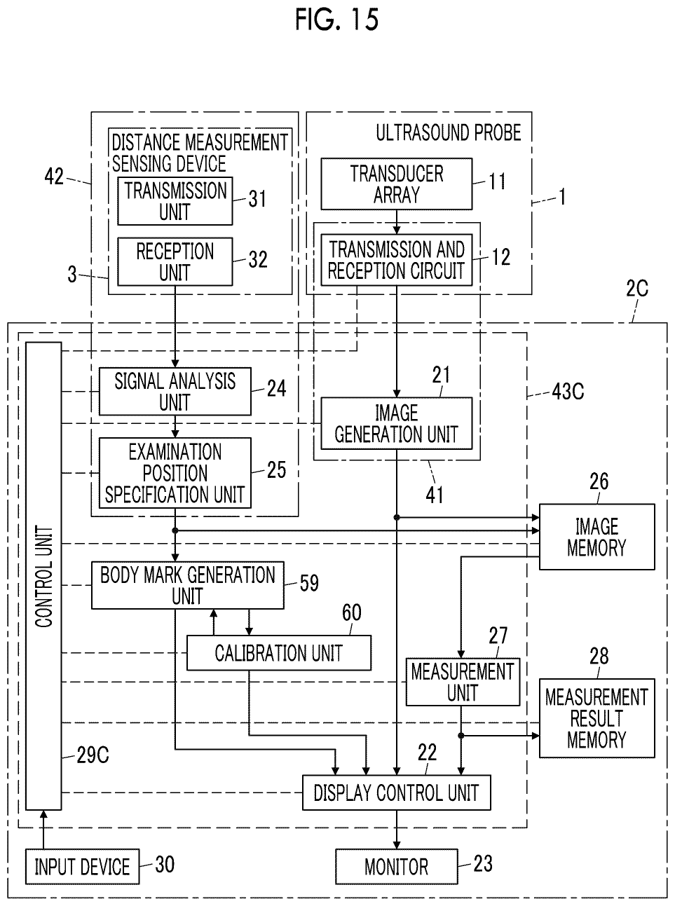

FIG. 15 shows a configuration of an ultrasound diagnostic apparatus according to Embodiment 4. The ultrasound diagnostic apparatus of Embodiment 4 comprises an apparatus body 2C instead of the apparatus body 2B with respect to the ultrasound diagnostic apparatus of Embodiment 3 shown in FIG. 8. In the apparatus body 2C, a calibration unit 60 is added, and a control unit 29C is provided instead of the control unit 29B, with respect to the apparatus body 2B in Embodiment 3.

In the apparatus body 2C, the calibration unit 60 is connected to the body mark generation unit 59 and the control unit 29C. In addition, the display control unit 22 is connected to the calibration unit 60. Further, the image generation unit 21, the display control unit 22, the signal analysis unit 24, the examination position specification unit 25, the measurement unit 27, the control unit 29, the body mark generation unit 59, and the calibration unit 60 constitute a processor 43C for the apparatus body 2C.

Here, it is known that a specific site such as the breast of the subject K generally has a different size, shape, position, and the like depending on an individual difference in a physique of the subject K.

In that respect, the calibration unit 60 corrects the deviation of the examination position 74 on the body mark caused by the individual difference in the physique of the subject K in order to accurately record the examination position on the body mark, tailored to the individual difference in the physique of the subject K. In this case, the calibration unit 60 can correct the deviation of the examination position 74 on the body mark by, for example, associating a plurality of positions predetermined on the body mark with the actual examination position on the subject K to be associated with the plurality of positions predetermined on the body mark, which is specified by the examination position specification unit 25.

The body mark generation unit 59 automatically records the examination position on the body mark by taking into account the deviation of the examination position 74 on the body mark, which is corrected by the calibration unit 60.

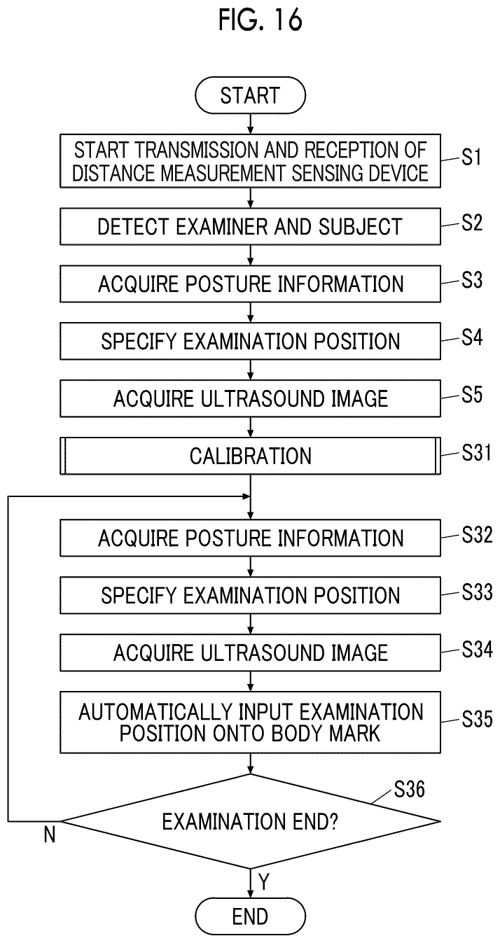

Next, the operation of the ultrasound diagnostic apparatus of Embodiment 4 will be described with reference to the flowchart shown in FIG. 16. Here, specifically, the operation of the ultrasound diagnostic apparatus in a case of examining the breast of the subject K will be described, but the examination position is not particularly limited to the breast of the subject K and may be, for example, the heart or the like.

In addition, the flowchart shown in FIG. 16 is a flowchart in which steps S6 and S7 are replaced with steps S31 to S36 with respect to the flowchart of Embodiment 1 shown in FIG. 5. Since steps S1 to S5 are the same as steps S1 to S5 in Embodiment 1, detailed descriptions thereof will not be repeated.

In addition, it is assumed that the body mark generation unit 59 stores in advance, as an initial setting, the body mark corresponding to the breast having a predetermined size, a predetermined shape, and a predetermined relative position, for example, for each site of the physique of a human being such as a head part, a shoulder part, and a waist part.

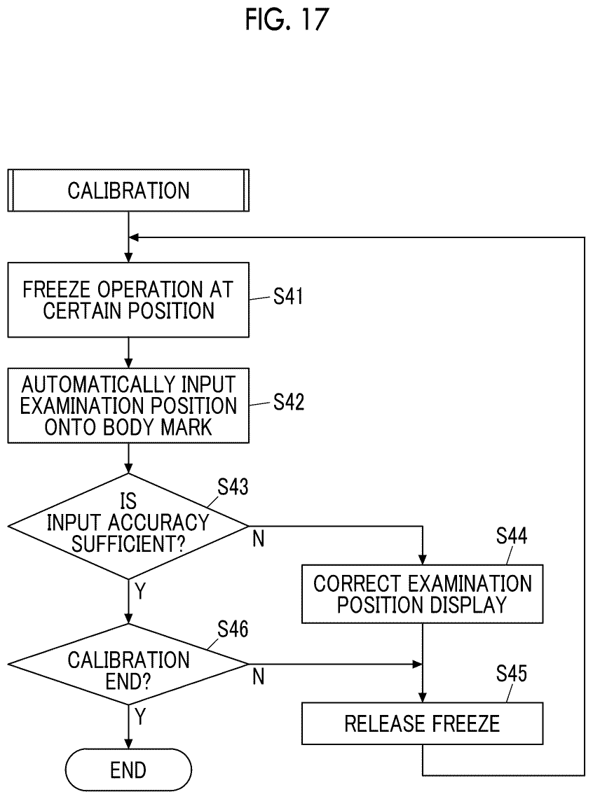

In a case where the ultrasound image of the breast of the subject K is acquired in step S5, the process proceeds to step S31. In step S31, the calibration unit 60 corrects the deviation of the examination position 74 on the body mark caused by the individual difference in the physique of the subject K. The calibration processing of step S31 is composed of the processing of steps S41 to S46 as shown in the flowchart of FIG. 17.

First, in step S41, the examiner J performs the freeze operation in a state in which the ultrasound probe 1 is brought into contact with a certain position on the breast of the subject K. In this case, the control unit 29C can display, for example, a message for bringing the ultrasound probe 1 into contact with a specific position, such as “please dispose the probe at the right end of the breast”, on the monitor 23. In this case, the examiner J brings the ultrasound probe 1 into contact with the subject K in accordance with the instruction displayed on the monitor 23.

In subsequent step S42, the body mark generation unit 59 automatically inputs the examination position, that is, the position of the ultrasound probe 1 on the subject K in a case where the freeze operation is performed in step S41, onto the body mark 71L or 71R of the breast.

In step S43, the calibration unit 60 determines whether or not the input accuracy of the examination position in step S42 is sufficient. Here, for example, since the size, the shape, and the position of the breast of the subject K vary depending on individual differences in the physique of the subject K, in a case where there is a deviation between the actual size, shape, and position of the breast of the subject K and the size, the shape, and the position of the breast corresponding to the body marks 71L and 71R stored by the body mark generation unit 59 as the initial setting, the input accuracy is determined to be insufficient. The calibration unit 60 can determine that the input accuracy of the examination position is sufficient, for example, in a case where the examination position automatically input onto the body mark 71L or 71R in step S42 and the corresponding position on the body mark 71L or 71R are within a predetermined distance, and can determine that the input accuracy of the examination position is insufficient in a case where the examination position and the corresponding position exceeds the predetermined distance.

In a case where it is determined in step S43 that the input accuracy of the examination position is insufficient, the process proceeds to step S44. In step S44, the calibration unit 60 corrects the examination position display by, for example, matching the examination position automatically input onto the body mark 71L or 71R in step S42 with the corresponding position on the body mark 71L or 71R.

In subsequent step S45, the control unit 29C releases the freeze. In a case where the processing of step S45 is completed, the process returns to step S41. In step S41, the examiner J brings the ultrasound probe 1 into contact with a different examination position on the same breast as the breast where ultrasound probe 1 is brought into contact in previous step S41, and performs a freeze operation.

After that, in step S42, the body mark generation unit 59 automatically inputs the examination position onto the same body mark 71L or 71R as the body mark 71L or 71R in previous step S42. Further, in step S43, the calibration unit 60 determines whether or not the input accuracy of the examination position automatically input in immediately preceding step S42 is sufficient.

In such a manner, as long as it is determined in step S43 that the input accuracy of the examination position is insufficient, the processing of steps S41 to S45 is repeated, and the actual size, shape, and position of the breast of the subject K and the size, shape, and position of the breast corresponding to the body mark 71L or 71R stored by the body mark generation unit 59 as the initial setting are associated with each other, and the deviation of the examination position on the body mark 71L or 71R caused by the individual difference in the physique of the subject K is corrected.

In a case where it is determined in step S43 that the input accuracy of the examination position is sufficient, the process proceeds to step S46. In step S46, the control unit 29 determines whether or not to end the calibration. The control unit 29 can determine to end the calibration, for example, in a case where the examiner J inputs an instruction to end the calibration via the input device 30, and can determine to continue the calibration in a case where no instruction to end the calibration is input.

In a case where it is determined in step S46 to continue the calibration, the freeze is released in step S45, and then the process returns to step S41, and the calibration processing is continued.

In a case where it is determined in step S46 to end the calibration, the calibration processing in step S31 ends.

By performing the calibration processing in such a manner, the examination position on the breast of the subject K can be accurately recorded on the body mark 71L or 71R.

In step S32 following step S31, the posture information of the examiner J and the subject K are acquired in the same manner as in step S3.

In step S33, the examination position is specified in the same manner as in step S4.

In step S34, the ultrasound image is acquired in the same manner as in step S5.

In step S35, the body mark generation unit 59 automatically inputs the examination position specified in step S33 onto the body mark 71L or 71R of the breast. Since the deviation of the examination position on the body mark 71L or 71R caused by the individual difference in the physique of the subject K is corrected in step S31, the body mark generation unit 59 can accurately input the examination position onto the body mark 71L or 71R of the breast.

In step S36, the control unit 29C determines whether or not to end the examination in the same manner as in step S7 of the flowchart of FIG. 14 in Embodiment 2. In a case where it is determined in step S36 to continue the examination, the processing returns to step S32, and the processing of steps S32 to S36 is sequentially performed. In a case where it is determined to end the examination in step S36, the operation of the ultrasound diagnostic apparatus following the flowchart of FIG. 16 ends.

As described above, with the ultrasound diagnostic apparatus of Embodiment 4, the calibration unit 60 corrects the deviation of the examination position on the body mark 71L or 71R caused by the individual difference in the physique of the subject K, so that the body mark generation unit 59 can accurately input the examination position onto the body mark 71L or 71R of the breast.

### EXPLANATION OF REFERENCES

1: ultrasound probe
2, 2A, 2B, 2C: apparatus body
3: distance measurement sensing device
11: transducer array
12: transmission and reception circuit
21: image generation unit
22: display control unit
23: monitor
24: signal analysis unit
25: examination position specification unit
26: image memory
27: measurement unit
28: measurement result memory
29, 29A, 29B, 29C: control unit
30: input device
31: transmission unit
32: reception unit
41: image acquisition unit
42: distance measurement device
43, 43A, 43B, 43C: processor
51: pulsar
52: amplification section
53: AD conversion section
54: beam former
55: signal processing section
56: DSC
57: image processing section
58: image acquisition condition setting unit
59: body mark generation unit
60: calibration unit
61, 71L, 71R: body mark
62, 74: examination position
73: axillary region
A: inner upper region
B: inner lower region
C: outer upper region
D: outer lower region
E1: shoulder part
E2: waist part
F: center line
J: examiner
K: subject
Q1, Q2: midpoint
T: examination table

What is claimed is:

1. An ultrasound diagnostic apparatus comprising:
a processor configured to specify an examination position of a subject by an examiner based on posture information of the examiner and the subject, which is acquired by analyzing reflection signals in a case where detection signals are transmitted from a distance measurement device to the examiner and the subject; and
a memory configured to store an ultrasound image of the subject and the examination position specified by the processor in association with each other.

2. The ultrasound diagnostic apparatus according to claim 1, further comprising:
a monitor; and
an ultrasound probe,
wherein the processor is configured to:
acquire the ultrasound image at the examination position of the subject by performing transmission and reception of an ultrasound beam using the ultrasound probe; and
display the ultrasound image on the monitor.

3. The ultrasound diagnostic apparatus according to claim 2,
wherein the processor is configured to display the specified examination position on the monitor.

4. The ultrasound diagnostic apparatus according to claim 3,
wherein the processor is configured to:
generate a body mark indicating the specified examination position; and
display the body mark on the monitor.

5. The ultrasound diagnostic apparatus according to claim 4,
wherein the processor is configured to correct a deviation of the examination position on the body mark caused by an individual difference in a physique of the subject.

6. The ultrasound diagnostic apparatus according to claim 4,
wherein the processor is configured to, upon that a freeze operation is performed by the examiner, automatically generate the body mark indicating the examination position and display the body mark on the monitor.

7. The ultrasound diagnostic apparatus according to claim 5,
wherein the processor is configured to, upon that a freeze operation is performed by the examiner, automatically generate the body mark indicating the examination position and display the body mark on the monitor.

8. The ultrasound diagnostic apparatus according to claim 3,
wherein the processor is configured to:
perform a measurement on the subject at the examination position, and
display a result of the measurement on the monitor.

9. The ultrasound diagnostic apparatus according to claim 4,
wherein the processor is configured to:
perform a measurement on the subject at the examination position, and
display a result of the measurement on the monitor.

10. The ultrasound diagnostic apparatus according to claim 5,
wherein the processor is configured to:
perform a measurement on the subject at the examination position, and
display a result of the measurement on the monitor.

11. The ultrasound diagnostic apparatus according to claim 2,
wherein the processor is configured to:
set an ultrasound image acquisition condition corresponding to the examination position; and
acquire the ultrasound image in accordance with the ultrasound image acquisition condition.

12. The ultrasound diagnostic apparatus according to claim 3,
wherein the processor is configured to:
set an ultrasound image acquisition condition corresponding to the examination position; and
acquire the ultrasound image in accordance with the ultrasound image acquisition condition.

13. The ultrasound diagnostic apparatus according to claim 4,
wherein the processor is configured to:
set an ultrasound image acquisition condition corresponding to the examination position; and
acquire the ultrasound image in accordance with the ultrasound image acquisition condition.

14. The ultrasound diagnostic apparatus according to claim 5,
wherein the processor is configured to:
set an ultrasound image acquisition condition corresponding to the examination position; and
acquire the ultrasound image in accordance with the ultrasound image acquisition condition.

15. The ultrasound diagnostic apparatus according to claim 11,
wherein the processor is configured to select the ultrasound image acquisition condition corresponding to the specified examination position from among a plurality of the ultrasound image acquisition conditions preset according to a plurality of the examination positions.

16. The ultrasound diagnostic apparatus according to claim 11,
wherein the ultrasound image acquisition condition includes at least one of an ultrasound beam depth, a focus position, or image processing.

17. The ultrasound diagnostic apparatus according to claim 15,
wherein the ultrasound image acquisition condition includes at least one of an ultrasound beam depth, a focus position, or image processing.

18. A control method of an ultrasound diagnostic apparatus, comprising:
specifying an examination position of a subject by an examiner based on posture information of the examiner and the subject, which is acquired by analyzing reflection signals in a case where detection signals are transmitted from a distance measurement device to the examiner and the subject; and
storing an ultrasound image of the subject and the specified examination position in a memory in association with each other.

19. A distance measurement device comprising:
a distance measurement sensing device configured to transmit detection signals and receive reflection signals with respect to an examiner and a subject; and
a processor configured to:
analyze the reflection signals received by the distance measurement sensing device to acquire posture information of the examiner and the subject; and
specify each of the examiner and the subject and specify an examination position of the subject by the examiner, based on the posture information.

20. The distance measurement device according to claim 19,
wherein the processor is configured to acquire the posture information of the examiner and the subject by using a machine learning model that has learned a reflection signal acquired by transmitting a detection signal to a human body by the distance measurement sensing device.
